(19) United States
(12) Patent Application Publication
(10) Pub. No.: US 2008/0004692 A1
(43) Pub. Date: Jan. 3, 2008

(54) Dynamically adjustable vascular stent

(76) Inventors: Michael R. Henson, Cota de Caza, CA (US); Shahram Moaddeb, Irvine, CA (US); Samuel M. Shaolian, Newport Beach, CA (US)

Correspondence Address: MCDERMOTT WILL & EMERY LLP, 18191 VON KARMAN AVE., SUITE 500, IRVINE, CA, 92612-7108, US

(21) Appl. No.: 11/477,152

(22) Filed: Jun. 28, 2006

(51) Int. Cl. A61F 2/06

(52) U.S. Cl. 623/1.18

(57) ABSTRACT

Methods and devices are provided for providing a protective framework for treating an aneurysm with embolic coils and preventing mitigation of the embolic coils from the aneurysm. A dynamically remodelable stent having a first and a second configuration is delivered into the blood vessel patient, such as a human or other animal, and positioned adjacent an ostium of an aneurysm while in the first, linear configuration. The dynamically remodelable stent may then be activated to assume a second, expanded configuration and thereby provide a protective framework spanning the neck of the aneurysm during and after delivery of embolic devices, such as embolic coils, to the aneurysm. The stent can be activated within the body of a patient in a minimally invasive or non-invasive manner such as by applying energy percutaneously or external to the patient's body. The energy may include, for example, acoustic energy, radio frequency energy, light energy and magnetic energy. In certain embodiments, the stent include a shape memory material that is responsive to changes in temperature and/or exposure to a magnetic field.

### BACKGROUND OF THE INVENTION

1. Field of the Invention

The present invention relates to methods and devices for treating aneurysms. More specifically, the present invention relates to vascular stents that can be adjusted within the body of a patient.

2. Description of the Related Art

Endovascular techniques for the treatment of intracranial aneurysms have evolved in the past two decades. Serbinenko reported his experience with endovascular techniques in 1979. when he described embolization with intravascular balloons. Balloon embolization became the endovascular procedure of choice in the 1980s. However, it was not ideally suited to selective occlusion of the aneurysm and preservation of the patency of the parent artery. Although it is sometimes possible to inflate a detachable balloon within the aneurysm while preserving flow through the parent artery, the disadvantage of this technique is that the size and shape of the balloon may not conform to that of the aneurysm, resulting in stretching of the aneurysm wall or incomplete filling of the aneurysm. The inability to customize a balloon to the configuration of an aneurysm led to the development of coil systems for aneurysm embolization.

Coil treatment permits conformation of the coil mass to the shape of the aneurysm, representing a significant improvement over balloon embolization. Initially, pushable coils were used for treatment of cerebrovascular lesions. The major disadvantage of this system was the inability to remove coils that did not assume a favorable position or configuration within the aneurysm.

This problem was addressed with the introduction of mechanically detachable and electrolytically detachable coils. First described by Guglielmi, et al for the experimental treatment of cerebrovascular lesions, electrolytically detachable coils were favored by clinical interventionists because of concerns about the forces applied within the aneurysm when detaching mechanically detachable coils. The Guglielmi Detachable Coil (GDC) design combines the advantages of soft compliant platinum with retrievability (a coil can be withdrawn, repositioned, or replaced before detachment), and atraumatic detachment.

Subsequent to the approval of the GDC (GDC, Boston Scientific/Target Therapeutics, Fremont, Calif.) by the FDA in 1995, there has been a trend toward the preferential use of endovascular therapy for the treatment of intracranial aneurysms. Early series reported use of GDC embolization solely for high-risk surgical cases (i.e., for patients of poor clinical grade or those with aneurysms deemed inoperable). Since that time, however, many centers have begun using endovascular treatment as first-line therapy for intracranial aneurysms. In particular, evidence of the efficacy of endovascular treatment for patients with subarachnoid hemorrhage presenting in poor clinical condition prompted some centers to adopt a policy of reserving the previous procedure of clip ligation to treat the aneurysms only for patients felt to be at high risk for complications from coil embolization.

At these centers, the anatomy of the aneurysm is evaluated with consideration for the ability to fill the aneurysm with coils without compromising the parent artery lumen. Favorable aneurysm anatomy includes a dome-to-neck ratio of greater than 2 mm and a small aneurysm neck diameter, usually less than 5 mm. In addition, aneurysm location may be a factor involved in treatment decisions. There have been lower rates of technical success for coil embolization for middle cerebral artery aneurysms. The size of the aneurysm dome and neck influences both the ability to occlude the aneurysm with coils and the rate of subsequent regrowth of the coil-treated aneurysm. The presence of a large intraparenchymal hematoma with mass effect may favor a decision to perform open surgery to reduce intracranial pressure. Conversely, evidence of significant brain swelling without a mass lesion may increase the risk of surgical retraction, resulting in reduction in local blood flow and ischemic injury. The overall trend has been to consider endovascular treatment first, reserving surgical therapy for aneurysms with unfavorable geometry, closeness to the cerebral convexities, or other surgical indications, such as intraparenchymal hematoma.

One of the major shortcomings of endovascular therapy, despite the widespread enthusiasm for its indications, was the inability to treat wide-necked aneurysms adequately. The propensity for coil herniation and parent vessel compromise made complete filling of the aneurysm nearly impossible and coil compaction or aneurysm regrowth a significant concern. Small aneurysms are normally treated with tiny coils that a doctor inserts into the aneurysm to fill and prevent it from bursting. However, with larger, or “wide-neck,” aneurysms—those more than 4 mm across—the “wide neck” prevents the coil from staying in place on its own and the coil has a tendency to slip through the opening and into the blood vessel. This “slippage” may cause recanalazation as well as a potentially dangerous thrombosis of the parent artery or distal embolization.

In recent years, however, researchers have described the treatment of wide-necked aneurysms with stent-assisted coiling in experimental models. Doctors can now use a flexible intracranial stent, which is folded up and sent to the necessary vessel in the brain through an artery in the leg. Once there, the stent opens up to support the walls of the blood vessel like a scaffold. It creates a blockage at the neck of the aneurysm. With this protection in place, coils may be packed more tightly within the aneurysm without fear of the coils slipping through the wide neck or parent vessel compromise, thereby reducing the risk for residual aneurysm or aneurysm regrowth. Therefore, more patients can undergo minimally invasive interventions to repair their cerebral aneurysms. However, some wide neck aneurysms in vessels deep within the brain require a narrow, tortuous path from the access site, typically the femoral artery, to the location of the aneurysm for treatment. Accordingly, what is needed is a stent that is adjustable such that it may assume a narrow configuration during delivery, but may be variably expandable once positioned over the aneurysm to provide protection against slippage of subsequently implanted embolization coils.

### SUMMARY OF THE INVENTION

Thus, it would be advantageous to develop an apparatus and methods for an dynamically remodeled stent that can be reconfigured within the body of a patient to provide a protective framework for implanting and maintaining one or more embolic devices within an aneurysm.

In one embodiment, disclosed is a method of treating an aneurysm within a patient, including providing a vascular stent comprising a shape memory material and having a first linear configuration and a second coiled configuration, advancing said vascular stent in said first linear configuration into a blood vessel proximal to an ostium of the aneurysm, positioning said stent adjacent the ostium of the aneurysm, and applying energy to the shape memory material of said vascular stent to change the stent from said first linear configuration into said second coiled configuration which at least partially spans the ostium of said aneurysm.

In another embodiment, a method for treating an aneurysm of a patient is disclosed including providing a vascular stent comprising a plurality of spaced-apart rings, each ring comprising a shape memory material and having a first configuration and a second configuration, wherein the cross-sectional diameter of the first configuration is smaller than the cross-sectional diameter of the second configuration. The stent is advanced into a blood vessel proximal to the ostium of the aneurysm with said rings in said first configuration and positioned adjacent the ostium of the aneurysm, such that said stent at least partially spans the ostium of said aneurysm. Energy is then applied to the shape memory material of at least one of said rings, thereby changing the rings from said first configuration into said second configuration.

In another embodiment, an adjustable shape-memory vascular stent is disclosed. The stent includes a body having distal and proximal ends and comprising a shape memory material, said body having a first linear configuration and a second coiled configuration, said body being changeable from said first configuration to said second configuration in response to an application of an activation energy to said shape memory material.

In another embodiment, an adjustable shape memory vascular stent is disclosed. The stent includes an elongate member with a plurality of rings spaced apart along the length of the elongate member, said rings comprising at least one shape memory material, said rings having a first compressed configuration and a second expanded configuration, said rings being changeable from said first configuration to said second configuration in response to an application of energy to said shape memory material.

In another embodiment, an adjustable shape-memory vascular stent includes means for stenting a blood vessel, the means comprising a shape memory material and having a first linear configuration and a second coiled configuration, the means for stenting being changeable from said first configuration to said second configuration in response to an application of an activation energy to the shape memory material.

For purposes of summarizing the invention, certain aspects, advantages and novel features of the invention have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIGS. 1A-1C illustrate variations of typical intracranial aneurysms.

FIG. 2 illustrates an embodiment of a dynamically remodeled stent in a first configuration attached to a delivery wire.

FIG. 3 illustrates the dynamically remodeled stent of FIG. 2 in a second configuration.

FIG. 4 illustrates the dynamically remodeled stent if FIG. 2 released from a delivery wire.

FIGS. 5A-5B are schematic representations of an embodiment of the dynamically remodeled stent being delivered adjacent to an aneurysm.

FIG. 6 is a schematic representation of the stent of FIG. 5 activated to assume a second configuration.

FIG. 7 is a schematic representation of embolic devices being delivered through the stent of FIGS. 5-6 to the aneurysm.

FIG. 8 is a schematic representation of the aneurysm of FIGS. 5-7 with the embolic devices and stent in place.

FIG. 9 is a schematic representation of the aneurysm of FIGS. 5-8 after the embolic devices thrombose and the stent has been removed.

### DETAILED DESCRIPTION OF THE PREFERRED EMBODIMENT

The present invention involves systems and methods for providing a dynamically remodelable vascular stent to provide a protective framework for treating aneurysms with embolic coils and preventing mitigation of the embolic coils from the aneurysms. In certain embodiments, a dynamically remodelable stent is delivered into the blood vessel patient such as a human or other animal, and positioned adjacent an aneurysm. The dynamically remodelable stent may be implanted percutaneously (e.g., via a femoral artery or vein, or other arteries or veins) as is known to someone skilled in the art. The dynamically remodelable stent is activated to assume an expanded shape and thereby provide a protective framework spanning the neck, or ostium, of the aneurysm during and after delivery of embolic devices, such as embolic coils, to the aneurysm. The embolic coils may then be delivered through the framework of the stent to the aneurysmal cavity in order to thrombose and occlude the aneurysm, thus preventing rupture of the aneurysmal wall.

In certain embodiments, the vascular stent may comprises a shape memory material that is responsive to changes in temperature and/or exposure to a magnetic field. Shape memory is the ability of a material to regain its shape after deformation. Shape memory materials include polymers, metals, metal alloys and ferromagnetic alloys. The vascular stent may be remodeled by applying an energy source to activate the shape memory material and cause it to change to a memorized shape. The energy source may include, for example, radio frequency (RF) energy, x-ray energy, microwave energy, ultrasonic energy such as focused ultrasound, high intensity focused ultrasound (HIFU) energy, light energy, electric field energy, magnetic field energy, combinations of the foregoing, or the like. For example, one embodiment of electromagnetic radiation that is useful is infrared energy having a wavelength in a range between approximately 750 nanometers and approximately 1600 nanometers. This type of infrared radiation may be produced efficiently by a solid state diode laser. In certain embodiments, the vascular stent may be selectively heated using short pulses of energy having an on and off period between each cycle. The energy pulses provide segmental heating which allows segmental adjustment of the vascular stent without adjusting the entire stent.

In certain embodiments, the vascular stent includes an energy absorbing material (also referred to herein as energy absorbing enhancement material) to increase heating efficiency and localize heating in the area of the shape memory material. Thus, damage to the surrounding tissue is reduced or minimized. Energy absorbing materials for light or laser activation energy may include nanoshells, nanospheres and the like, particularly where infrared laser energy is used to energize the material. Such nanoparticles may be made from a dielectric, such as silica, coated with an ultra thin layer of a conductor, such as gold, and be selectively tuned to absorb a particular frequency of electromagnetic radiation. In certain such embodiments, the nanoparticles range in size between about 5 nanometers and about 20 nanometers and can be suspended in a suitable material or solution, such as saline solution. Coatings comprising nanotubes or nanoparticles can also be used to absorb energy from, for example, HIFU, MRI, inductive heating, or the like.

In other embodiments, thin film deposition or other coating techniques such as sputtering, reactive sputtering, metal ion implantation, physical vapor deposition, and chemical deposition can be used to cover portions or all of the vascular stent. Such coatings can be either solid or microporous. When HIFU energy is used, for example, a microporous structure traps and directs the HIFU energy toward the shape memory material. The coating improves thermal conduction and heat removal. In certain embodiments, the coating also enhances radio-opacity of the vascular stent. Coating materials can be selected from various groups of biocompatible organic or non-organic, metallic or non-metallic materials such as Titanium Nitride (TiN), Iridium Oxide (Irox), Carbon, Platinum black, Titanium Carbide (TiC) and other materials used for pacemaker electrodes or implantable pacemaker leads. Other materials discussed herein or known in the art can also be used to absorb energy.

In addition, or in other embodiments, fine conductive wires such as platinum coated copper, titanium, tantalum, stainless steel, gold, or the like, are wrapped around the shape memory material to allow focused and rapid heating of the shape memory material while reducing undesired heating of surrounding tissues.

In certain embodiments, the energy source is applied surgically either during implantation of the stent or at a later time. For example, the shape memory material can be heated during implantation of the stent by touching the stent with a warm object. As another example, the energy source can be surgically applied after the stent has been implanted by percutaneously inserting a catheter into the patient's body and applying the energy through the catheter. For example, RF energy, light energy or thermal energy (e.g., from a heating element using resistance heating) can be transferred to the shape memory material through a catheter positioned on or near the shape memory material. Alternatively, thermal energy can be provided to the shape memory material by injecting a heated fluid through a catheter or circulating the heated fluid in a balloon through the catheter placed in close proximity to the shape memory material. As another example, the shape memory material can be coated with a photodynamic absorbing material which is activated to heat the shape memory material when illuminated by light from a laser diode or directed to the coating through fiber optic elements in a catheter. In certain such embodiments, the photodynamic absorbing material includes one or more drugs that are released when illuminated by the laser light.

As discussed above, shape memory materials include, for example, polymers, metals, and metal alloys including ferromagnetic alloys. Exemplary shape memory polymers that are usable for certain embodiments of the present invention are disclosed by Langer, et al. in U.S. Pat. No. 6,720,402, issued Apr. 13, 2004, U.S. Pat. No. 6,388,043, issued May 14, 2002, and U.S. Pat. No. 6,160,084, issued Dec. 12, 2000, each of which are hereby incorporated by reference herein. Shape memory polymers respond to changes in temperature by changing to one or more permanent or memorized shapes. In certain embodiments, the shape memory polymer is heated to a temperature between approximately 38 degrees Celsius and approximately 60 degrees Celsius. In certain other embodiments, the shape memory polymer is heated to a temperature in a range between approximately 40 degrees Celsius and approximately 55 degrees Celsius. In certain embodiments, the shape memory polymer has a two-way shape memory effect wherein the shape memory polymer is heated to change it to a first memorized shape and cooled to change it to a second memorized shape. The shape memory polymer can be cooled, for example, by inserting or circulating a cooled fluid through a catheter.

Shape memory polymers implanted in a patient's body can be heated non-invasively using, for example, external light energy sources such as infrared, near-infrared, ultraviolet, microwave and/or visible light sources. Preferably, the light energy is selected to increase absorption by the shape memory polymer and reduce absorption by the surrounding tissue. Thus, damage to the tissue surrounding the shape memory polymer is reduced when the shape memory polymer is heated to change its shape. In other embodiments, the shape memory polymer comprises gas bubbles or bubble containing liquids such as fluorocarbons and is heated by inducing a cavitation effect in the gas/liquid when exposed to HIFU energy. In other embodiments, the shape memory polymer may be heated using electromagnetic fields and may be coated with a material that absorbs electromagnetic fields.

Certain metal alloys have shape memory qualities and respond to changes in temperature and/or exposure to magnetic fields. Exemplary shape memory alloys that respond to changes in temperature include titanium-nickel, copper-zinc-aluminum, copper-aluminum-nickel, iron-manganese-silicon, iron-nickel-aluminum, gold-cadmium, combinations of the foregoing, and the like. In certain embodiments, the shape memory alloy comprises a biocompatible material such as a titanium-nickel alloy.

Shape memory alloys exist in two distinct solid phases called martensite and austenite. The martensite phase is relatively soft and easily deformed, whereas the austenite phase is relatively stronger and less easily deformed. For example, shape memory alloys enter the austenite phase at a relatively high temperature and the martensite phase at a relatively low temperature. Shape memory alloys begin transforming to the martensite phase at a start temperature (Mₛ) and finish transforming to the martensite phase at a finish temperature (M_{f}). Similarly, such shape memory alloys begin transforming to the austenite phase at a start temperature (Aₛ) and finish transforming to the austenite phase at a finish temperature (A_{f}). Both transformations have a hysteresis. Thus, the Mₛ temperature and the A_{f} temperature are not coincident with each other, and the M_{f} temperature and the Aₛ temperature are not coincident with each other.

In certain embodiments, the shape memory alloy is processed to form a memorized shape in the austenite phase in the form of a coil or coil portion. The shape memory alloy is then cooled below the M_{f} temperature to enter the martensite phase and deformed into a linear portion. For example, in certain embodiments, the shape memory alloy is formed into a linear wire or ribbon that has a smaller cross-sectional diameter than the memorized tubular or coiled shape to better facilitating delivery of the stent through a narrow tortuous path in the neurovasculature. After the wire is delivered to the aneurysm site, the wire may non-invasively adjusted or remodeled to assume a tubular or coiled stent formation spanning the neck of the aneurysm by heating the shape memory alloy to an activation temperature (e.g., temperatures ranging from the Aₛ temperature to the A_{f} temperature).

Thereafter, when the shape memory alloy is exposed to a temperature elevation and transformed to the austenite phase, the alloy changes in shape from the deformed shape to the memorized shape. Activation temperatures at which the shape memory alloy causes the shape of the stent to change shape can be selected and built into the stent such that collateral damage is reduced or eliminated in tissue adjacent the stent during the activation process. Exemplary A_{f} temperatures for suitable shape memory alloys range between approximately 45 degrees Celsius and approximately 70 degrees Celsius. Furthermore, exemplary Mₛ temperatures range between approximately 10 degrees Celsius and approximately 20 degrees Celsius, and exemplary M_{f} temperatures range between approximately −1 degrees Celsius and approximately 15 degrees Celsius. The size of the stent can be changed all at once or incrementally in small steps at different times in order to achieve the adjustment necessary to produce the desired clinical result.

Certain shape memory alloys may further include a rhombohedral phase, having a rhombohedral start temperature (Rₛ) and a rhombohedral finish temperature (R_{f}), that exists between the austenite and martensite phases. An example of such a shape memory alloy is a NiTi alloy, which is commercially available from Memry Corporation (Bethel, Conn.). In certain embodiments, an exemplary Rₛ temperature range is between approximately 30 degrees Celsius and approximately 50 degrees Celsius, and an exemplary R_{f} temperature range is between approximately 20 degrees Celsius and approximately 35 degrees Celsius. One benefit of using a shape memory material having a rhombohedral phase is that in the rhomobohedral phase the shape memory material may experience a partial physical distortion, as compared to the generally rigid structure of the austenite phase and the generally deformable structure of the martensite phase.

Certain shape memory alloys exhibit a ferromagnetic shape memory effect wherein the shape memory alloy transforms from the martensite phase to the austenite phase when exposed to an external magnetic field. The term “ferromagnetic” as used herein is a broad term and is used in its ordinary sense and includes, without limitation, any material that easily magnetizes, such as a material having atoms that orient their electron spins to conform to an external magnetic field. Ferromagnetic materials include permanent magnets, which can be magnetized through a variety of modes, and materials, such as metals, that are attracted to permanent magnets. Ferromagnetic materials also include electromagnetic materials that are capable of being activated by an electromagnetic transmitter, such as one located outside the body. Furthermore, ferromagnetic materials may include one or more polymer-bonded magnets, wherein magnetic particles are bound within a polymer matrix, such as a biocompatible polymer. The magnetic materials can comprise isotropic and/or anisotropic materials, such as for example NdFeB (Neodynium Iron Boron), SmCo (Samarium Cobalt), ferrite and/or AlNiCo (Aluminum Nickel Cobalt) particles.

Thus, a stent comprising a ferromagnetic shape memory alloy can be delivered in a first configuration having a first shape and later changed to a second configuration having a second (e.g., memorized) shape without heating the shape memory material above the Aₛ temperature. Advantageously, nearby healthy tissue is not exposed to high temperatures that could damage the tissue. Further, since the ferromagnetic shape memory alloy does not need to be heated, the size of the stent can be adjusted more quickly and more uniformly than by heat activation.

Exemplary ferromagnetic shape memory alloys include Fe—C, Fe—Pd, Fe—Mn—Si, Co—Mn, Fe—Co—Ni—Ti, Ni—Mn—Ga, Ni₂MnGa, Co—Ni—Al, and the like. Certain of these shape memory materials may also change shape in response to changes in temperature. Thus, the shape of such materials can be adjusted by exposure to a magnetic field, by changing the temperature of the material, or both.

In certain embodiments, combinations of different shape memory materials are used. For example, stents according to certain embodiments comprise a combination of shape memory alloys having different activation temperatures. In certain such embodiments, the stent may be activated from its linear delivery configuration to one or more intermediate coil configurations of varying cross-sectional diameters to provide greater flexibility in customizing the stent for variable sized blood vessel In addition, or in other embodiments, shape memory polymers are used with shape memory alloys to create a bi-directional (e.g., capable of expanding and contracting) stent. Bi-directional stents can be created with a wide variety of shape memory material combinations having different characteristics.

In the following description, reference is made to the accompanying drawings, which form a part hereof, and which show, by way of illustration, specific embodiments or processes in which the invention may be practiced. Where possible, the same reference numbers are used throughout the drawings to refer to the same or like components. In some instances, numerous specific details are set forth in order to provide a thorough understanding of the present disclosure. The present disclosure, however, may be practiced without the specific details or with certain alternative equivalent components and methods to those described herein. In other instances, well-known components and methods have not been described in detail so as not to unnecessarily obscure aspects of the present disclosure.

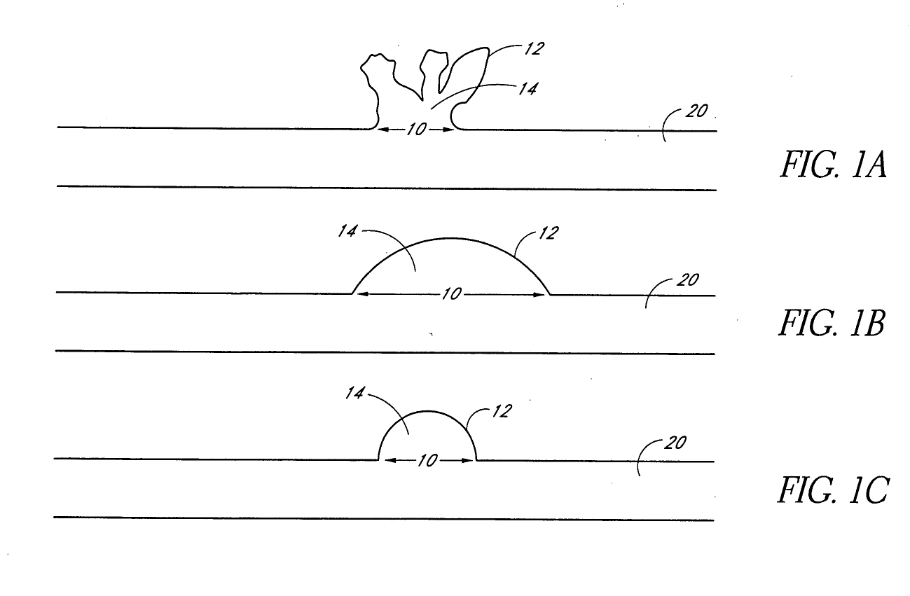

With reference to FIGS. 1A-1C, various types of wide necked aneurysms are shown. Typically, a wide neck aneurysm has a neck, or ostium, 10 greater than 4 mm across or a dome 12 to neck 10 ratio of less than 2. These aneurysms were previously considered untreatable because the wide neck prevents embolic coils from remaining in position in the aneurysm sac 14 and increases the risk of the coils migrating into the blood vessel 20.

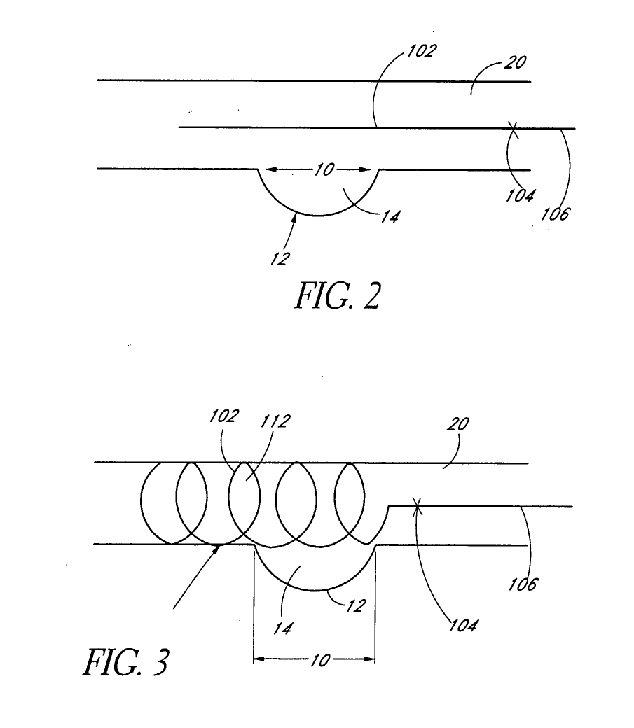

FIGS. 2-4 show one embodiment of a dynamically adjustable stent 100 that can be remodeled after delivery to an aneurysm site. The path through the vasculature to an aneurysm, especially a deeply located cerebral aneurysm is often narrow and tortuous. Accordingly, a stent with a small delivery size is advantageous. As shown in FIG. 2, the stent 100 comprises a continuous, flat wire or ribbon 102 comprised at least in part of a shape memory material, such as NiTinol or any other suitable shape memory metal alloy or polymer as discussed above. In the first, delivery configuration of the stent 102, the wire is stretched out linearly to form a narrow cross-section. The wire 102 may be of a suitable length to subsequently, upon activation, form a coiled stent of a desired cross section and length with a desired spacing between the coils. For example, the length of the wire may be about 15-40 mm such that when activated, the wire may assume a coiled configuration having a length between about 10-20 mm and a cross-sectional diameter between about 2-6 mm. The spacing between coils must be sufficient for delivery of an embolic device, such as one or more embolic coils, to the aneurysm sac via a micro-catheter.

However, in the delivery configuration, as shown in FIG. 2, the cross-sectional diameter of the stent 102 in is the same as the cross-sectional diameter of the wire 102, for example 0.05-0.5 mm. Therefore, the stent 100 may be delivered in a minimally invasive percutaneous manner through a delivery catheter having a small cross-sectional diameter. The wire 102 has a release point 104 at its proximal end for releasing the wire 102 from a delivery wire 106 once the wire 102 has been activated. The release point may comprise a severable mechanical connection such as interlocking notches and grooves, or an electrical connection. The delivery wire 106 comprises a pusher wire for advancing the stent wire 102 through a delivery catheter to the aneurysm site.

As shown in FIG. 3, once the wire 102 has been positioned across the neck 10 of the aneurysm 12, the wire 102 may be activated to assume a second, implanted configuration. The implanted configuration is preferably a continuous, helical coiled configuration with the coils having a cross-sectional diameter such that the coils radially exert pressure against the side walls of the blood vessel, thereby securing the position of the stent in the blood vessel. For example, depending on the location of the aneurysm and the size of the blood vessel, the coiled configuration may have a cross-sectional diameter of between about 2-20 mm. The wire is preferably activated by applying energy to the wire to heat the shape memory material to its austenite transition temperature and thereby cause the shape memory material to assume its preformed austenite shape, as discussed above.

Preferably, the wire 102 comprises a shape memory material that responds to the application of temperature that differs from a nominal ambient temperature, such as the nominal body temperature of 37° Celsius for humans. For example, exemplary A_{f} temperatures for the shape memory material of the wire 102 at which substantially maximum expansion occurs are in a range between approximately 38° Celsius and 75° Celsius, alternatively between approximately 39° Celsius and 75° Celsius.

In certain embodiments, the activation energy may comprise an RF activation energy that can be applied by means of either a detachable electrode attached to the wire or by a separate catheter that can be placed in contact with the wire, as will be discussed on more detail below. Alternatively, the activation energy may comprise light energy, or thermal energy as discussed above.

In certain embodiments, the wire 102 may comprise a single shape memory material that is pre-trained to assume the helical coil configuration as the temperature of the material reaches an austenite transition temperature. Alternatively, the wire may comprise a plurality of alternating sections of shape memory material and a second material, wherein the shape memory sections are configured to cause the wire 102 to assume the helical coil configuration as the temperature of the wire 102 reaches an austenite transition temperature.

In certain embodiments, the wire 102 may initially expand to a coiled configuration having a first cross-sectional diameter as the temperature nears the starting austenite transition temperature, Aₛ. Then, as the temperature continues to increase beyond the starting austenite temperature, the coiled configuration may continue to expand in cross-sectional diameter. Here, the cross-sectional diameter of the final implanted configuration may be incrementally expanded to accommodate a range of vessel diameters by gradually or incrementally increasing the temperature of the wire 102 and stopping once the desired cross-section of the coiled stent is achieved.

For example, the wire 102 may be configured to respond by starting to contract and coil upon heating the wire 102 above the Aₛ temperature of the shape memory material and continuing to incrementally expand the cross-sectional diameter of the coils as the temperature is firther increased to the A_{f} temperature. For example, in certain embodiments, the shape memory material may have a threshold transition temperature of about 38° C. wherein the shape memory material begins to transition, but may still continue to expand as the temperature increases to 75° C. wherein the final, preformed austenite shape is fully realized.

In certain embodiments, the temperature may be raised in one or more pre-determined increments to incrementally increase the cross-sectional diameter of the coiled stent in pre-determined increments. Alternatively, the temperature may be raised gradually to continuously and gradually increase the cross-sectional diameter of the coiled stent until the desired cross-sectional diameter is reached.

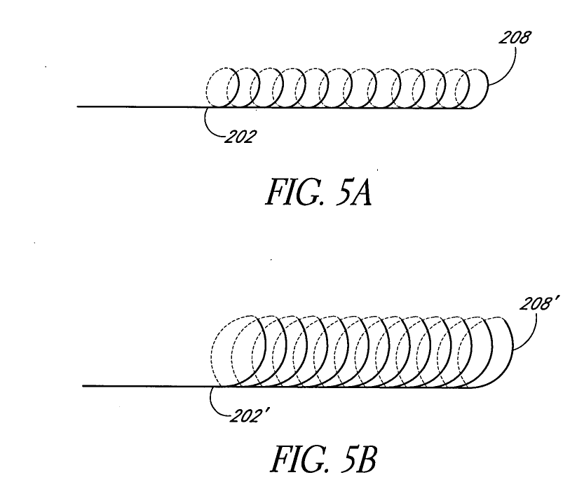

In an alternative embodiment, as shown in FIG. 5, the stent may comprise an elongate wire 202 having a plurality of adjustable coils 208 spaced apart along the length of the wire 202. For example, in certain embodiments, the elongate wire may be about 10 mm, alternatively about 15 mm, alternatively about 20 mm in length or any length suitable for spanning the neck of the aneurysm. The coils are preferably spaced apart a distance that permits the delivery of one or more embolic coils to the aneurysm sac through a micro-catheter positioned between the coils 208, while at the same time provides a framework that prevents subsequent migration of the coils from the aneurysm sac into the blood vessel. Each of the coils 208 may comprise a shape memory material, such as an NiTi wire or any other suitable shape memory metal alloy or polymer discussed above. Each of the coils 208 have a first, martensite configuration comprising a contracted coil with a small cross-sectional diameter and a second, austenite configuration having an expanded cross-sectional diameter. For example, in certain embodiments, the coils 208 may expand by percentage in a range between approximately 5% and 50% or more, where the percentage of change is defined as a ratio of the difference between the starting cross section and finish cross-section divided by the starting cross section.

The coils 208 are configured to be delivered through the patient's vasculature to the aneurysm site in the first contracted configuration, shown in FIG. 5 and then upon application of an activation energy sufficient to raise the temperature of the coils to the austenite transition temperature, the coils 208 will assume the expanded austenite configuration, shown in FIG. 5A. As discussed above, the shape memory material of the coils 208 may be selected such that the austenite transition occurs gradually over a temperature range such that the expansion of the coil diameter may be incrementally controlled by incrementally increasing the temperature of the coils 208. Here, each of the coils may be simultaneously expanded, for example by simultaneous application of an activation energy to each of the coils. Alternatively, the coils 208 may be sequentially expanded, for example starting with the proximal end and progressing toward the distal coil, or alternatively starting with the distal coil and progressing toward the proximal coil. In an alternative embodiment, each of the coils 208 may comprise alternating segments of shape memory material and an insulating material such that the cross-sectional diameter of the coil 208 may be adjusted by activating more or less shape memory segments on the coil 208.

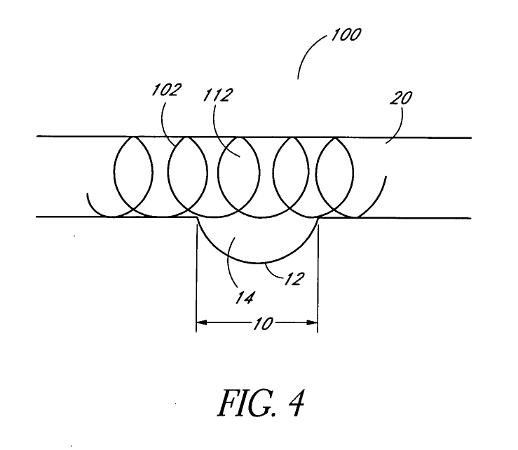

As shown in FIG. 4, once the wire 102 has assumed its implanted, coiled configuration and is firmly anchored in position against the walls of the blood vessel 20, the release point 104 may be engaged to release the wire 102 from the delivery wire 106. The release point may be engaged by application of energy to the release point, by a mechanical means, or any other suitable means known in the arts. Activation of the release point will detach the stent 100 in place at the aneurysm site. In certain embodiments, the release point is configured such that the proximal end of the wire 102 may be re-engaged by the delivery wire 106, for example to remove the wire 102 from the blood vessel 20 once the embolic devices have thrombosed.

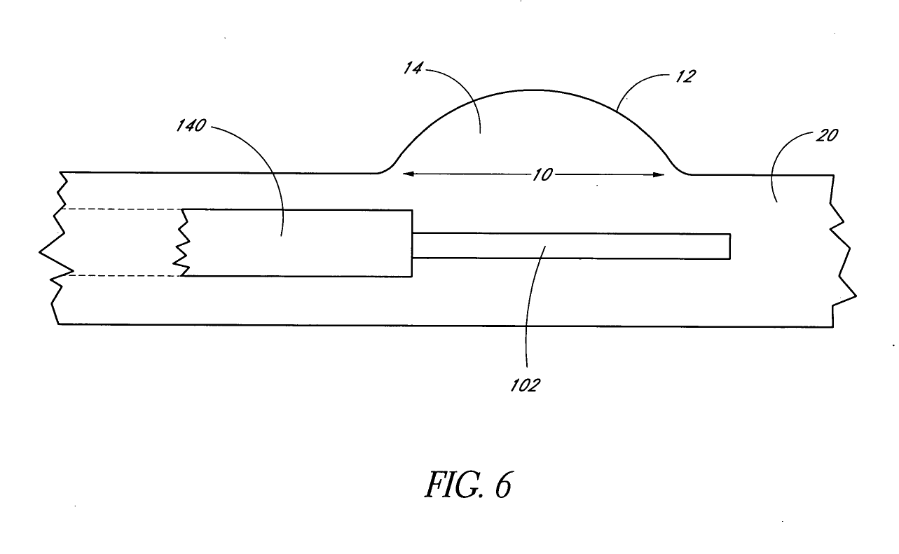

In use, as shown in FIGS. 6-9, a delivery catheter 140 may be advanced through a patient's blood vessel 20 proximal to the aneurysm 10 using methods known in the art. Preferably, the delivery catheter 140 has a small cross-sectional diameter, for example about 4 mm or less, such that it can be advanced through the small diameter neurovasculature to the site of a cerebral aneurysm. Once the delivery catheter 140 has been positioned at the aneurysm 10, the vascular stent may be advanced through the delivery catheter 140 and out the distal end of the catheter to the aneurysm 10. Here, the vascular stent 100 is configured in its first, delivery shape as an elongate wire 102 with a cross-section equal to the cross section of the wire 102. The proximal end of the wire 102 is attached to a delivery wire 106 for pushing the wire 102 through the delivery catheter 140 and positioning the wire 102 such that when the wire 102 expands to its coiled configuration 112, the vascular stent 100 will extend beyond the proximal and distal ends of the aneurysm neck 10.

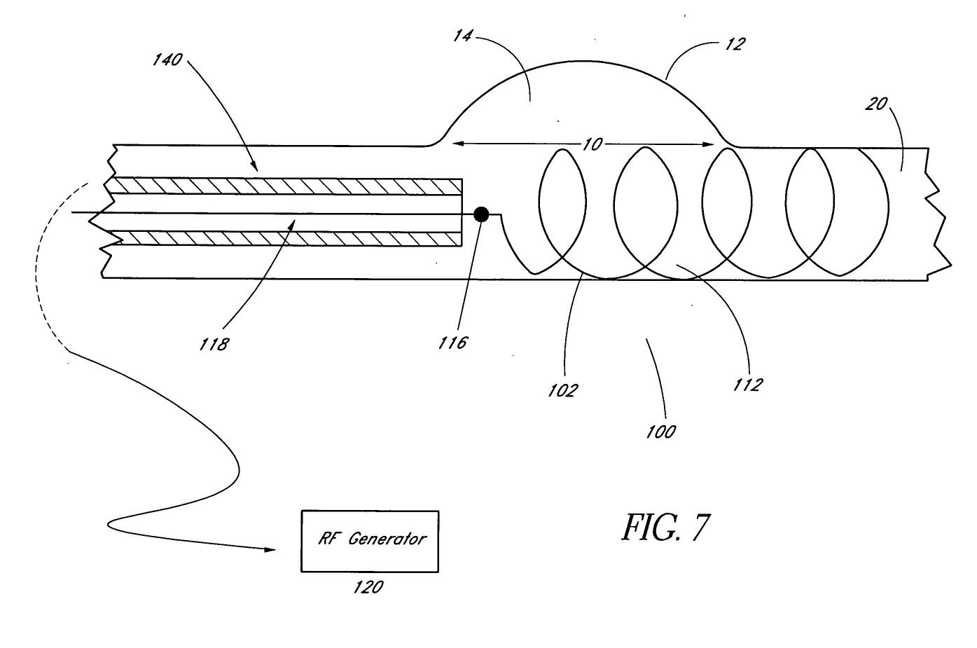

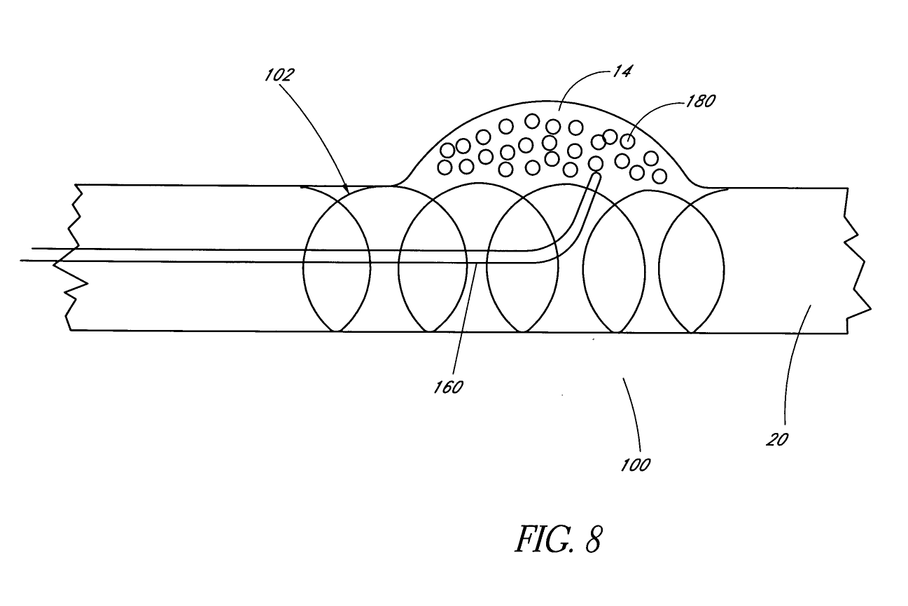

Once the wire 102 has been properly positioned adjacent the aneurysm 12, an RF energy may be applied to the wire 102 to raise the temperature of the wire 102 to the austenite temperature, thereby causing the wire 102 to assume a second coiled configuration 112 comprising a plurality of helical coils anchored against the side walls of the blood vessel 20. As shown in FIG. 7, the RF energy may be applied by an RF electrode 116 located at the proximal end of the wire stent 102. The RF electrode 116 may be connected to an RF lead wire 118 which extends proximally through the delivery catheter 140 to an RF generator 120 located outside of the patient. Alternatively, the RF generator 120 may be attached to the proximal end of the delivery wire 106 and the RF energy may be delivered to the wire stent 102 through the delivery wire 106. In alternative embodiments, the RF energy may be applied by a separate probe which is advanced through the delivery catheter 140 until it contacts the RF electrode 116 on the wire stent 102 to apply the RF energy. In an alternative embodiment, the RF energy may be applied in a non-invasive manner from outside the body. For example, as discussed above, a magnetic field and/or RF pulses can be applied to a wire 102 within a patient's body with an apparatus external to the patient's body such as is commonly used for magnetic resonance imaging (MRI).

As shown in FIG. 7, the wire 102 may have a single RF electrode 116 located at the proximal end such that the RF energy is applied to the RF electrode 116 simultaneously raises the temperature of the entire length of the wire 102 and thereby causes the entire length of the wire to simultaneously and uniformly undergo a shape transition from the elongate configuration to a coiled configuration. Alternatively, the wire 102 may have a plurality of RF electrodes spaced apart along the length of the wire 102. For example, as shown in FIG. 5, the wire 202 may comprise a plurality of segmented rings 208 spaced apart along the length, each ring having a separate RF electrode. Here, an RF probe may be advanced through the delivery catheter and along the wire to individually and sequentially apply RF energy to each separate ring. Thus, the rings 208 may be deployed in a sequential fashion, for example from the distal end first, or alternatively from the proximal end first. In addition, the cross-sectional diameter of each ring may be individually tailored to the diameter of the blood vessel at that point.

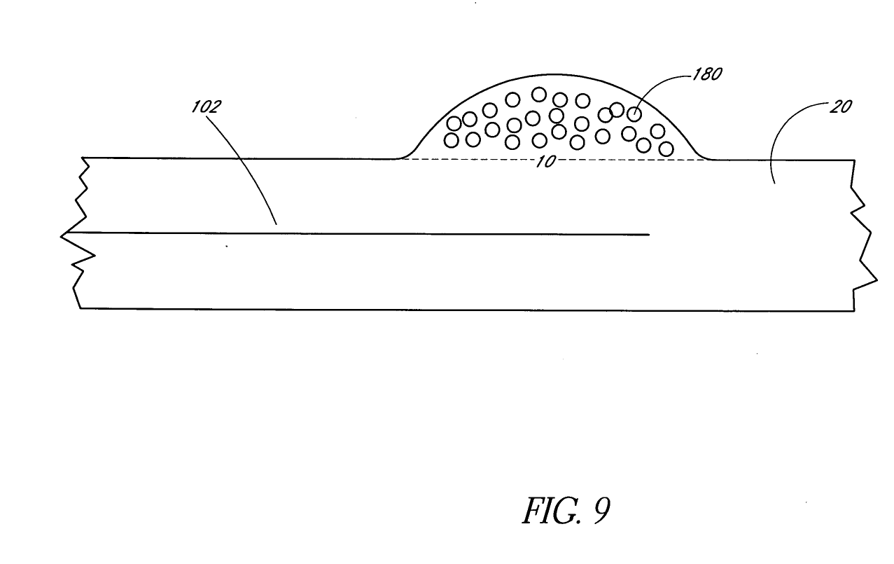

As shown in FIG. 8, once the stent 100 has been placed across the aneurysm neck 10, a microcatheter 160 may be navigated through the stent 100 and in between two stent coils into the aneurysm sac 14. The microcatheter may deliver one or more embolic devices, such as embolic coils 180, to the aneurysm sac 14 in order to completely fill the aneurysm sac 14. The stent 100 provides a scaffold preventing the coils 180 from migrating out of the wide neck 10 of the aneurysm 12. Typically, within about 30-60 minutes, blood clots around the embolic coils and the coils become incorporated into the aneurysm, sealing off the aneurysm from the blood flow in the parent blood vessel and anchoring the coils within the aneurysm sac 14. Once the aneurysm 12 is sealed off, the vascular stent 100 is no longer necessary to provide a protective scaffold for preventing migration of the embolic coils. Thus, as shown in FIG. 9, in certain embodiments, the vascular stent 100 may be transformed a second time to assume its initial configuration as an elongate wire 102 and may then be removed from the blood vessel 20. The stent 100 may be transformed from the expanded coil configuration to its initial linear configuration by reactivating the wire 102 at a second, different transition temperature. Some shape memory alloys, such as NiTi or the like, respond to the application of a temperature below the nominal ambient temperature. After the expansion cycle has been performed, the wire 102 may be cooled below the M_{f} temperature to finish the transformation to the martensite phase and reverse the expansion cycle. As discussed above, certain polymers also exhibit a two-way shape memory effect and can be used to both coil and extend the wire 102 through heating and cooling processes. Cooling can be achieved, for example, by inserting a cool liquid onto or into the stent 100 through a catheter, or by cycling a cool liquid or gas through a catheter placed near the stent 100. Exemplary temperatures for a NiTi embodiment for cooling and reversing a coil expansion cycle range between approximately 20° Celsius and approximately 30° Celsius.

Once the stent 100 has been transformed to its original shape as an elongate wire 102, the delivery wire 106 may be reattached to the release point 104 and used to pull the wire 102 proximally through the delivery catheter 140 and thereby withdraw it from the patient's blood vessel 20. This will eliminate the need of having a long-term stent in place and reduce the possibility of stenosis downstream due to the radial pressure from the stent against the blood vessel walls.

While certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A method for treating an aneurysm of a patient comprising:
providing a vascular stent comprising a shape memory material and having a first linear configuration and a second coiled configuration;
advancing said vascular stent in said first linear configuration into a blood vessel proximal to an ostium of the aneurysm;
positioning said stent adjacent the ostium of the aneurysm; and
applying energy to the shape memory material of said vascular stent, thereby changing the stent from said first linear configuration into said second coiled configuration, wherein the stent in said coiled configuration at least partially spans the ostium of said aneurysm.

2. The method of claim 1, further comprising:
introducing an embolization element into said aneurysm between adjacent windings or loops of said vascular stent.

3. The method of claim 2, wherein said embolization element comprises one or more embolic coils.

4. The method of claim 2, further comprising:
activating the shape memory material of said vascular stent to reassume a substantially linear configuration; and
withdrawing said vascular stent from said blood vessel.

5. The method of claim 1, wherein said applying energy comprises heating said shape memory material of said vascular stent, wherein said shape memory material changes shape in response to being heated.

6. The method of claim 5 wherein said shape memory material assumes said second coiled configuration in response to being heated.

7. The method of claim 5, wherein said applying energy further comprises raising a temperature of said shape memory material beyond a second temperature and wherein said shape memory material further changes shape in response to being heated above said second temperature.

8. The method of claim 7, wherein a cross-section of said coiled configuration increases in response to said shape memory material being heated beyond said second temperature.

9. The method of claim 1, wherein the applying energy comprises applying at least one of magnetic resonance imaging energy, high-intensity focused ultrasound energy, radio frequency (RF) energy, x-ray energy, microwave energy, light energy, electric field energy, magnetic field energy, inductive heating, and conductive heating.

10. The method of claim 1, wherein the applying energy comprises applying RF energy to the shape memory material.

11. The method of claim 1, further comprising advancing a delivery catheter having distal and proximal ends and a lumen extending therethrough through said blood vessel proximal to said aneurysm, and wherein advancing said vascular stent further comprises advancing said vascular stent through said lumen of said delivery catheter.

12. The method of claim 11 wherein advancing said vascular stent further comprises:
coupling the vascular stent to an elongate delivery member; and
advancing said delivery member through said lumen of said delivery catheter.

13. The method of claim 11, wherein the vascular stent is connected to an RF lead wire extending proximally through said lumen in said delivery catheter, and wherein applying said energy comprises coupling said RF lead wire to an RF generator located outside of the patient.

14. The method of claim 11, wherein said applying energy further comprises advancing an RF probe through the delivery catheter and contacting the RF probe to said vascular stent to deliver RF energy to said shape memory material.

15. A method for treating an aneurysm of a patient comprising:
providing a vascular stent comprising a plurality of spaced-apart rings, each ring comprising a shape memory material and having a first configuration and a second configuration, wherein the cross-sectional diameter of the first configuration is smaller than the cross-sectional diameter of the second configuration;
advancing said vascular stent into a blood vessel proximal to the ostium of the aneurysm with said rings in said first configuration;
positioning said stent adjacent the ostium of the aneurysm, such that said stent at least partially spans the ostium of said aneurysm; and
applying energy to the shape memory material of at least one of said rings, thereby changing the rings from said first configuration into said second configuration.

16. The method of claim 15, further comprising:
introducing one or more embolization elements into said aneurysm between adjacent rings of said vascular stent.

17. The method of claim 16, further comprising:
activating the shape memory material of said rings to reassume substantially said first configuration; and
withdrawing said vascular stent from said blood vessel.

18. The method of claim 15, wherein the applying energy comprises heating said shape memory material of said rings to a temperature, wherein said shape memory material changes shape in response to being heated to said temperature.

19. The method. of claim 18, wherein the applying energy comprises simultaneously applying energy to each of said plurality of rings.

20. The method of claim 18, wherein the applying energy comprises sequentially applying energy to each of said plurality of rings.

21. The method of claim 15, wherein the applying energy comprises applying at least one of magnetic resonance imaging energy, high-intensity focused ultrasound energy, radio frequency energy, x-ray energy, microwave energy, light energy, electric field energy, magnetic field energy, inductive heating, and conductive heating to the shape memory material.

22. The method of claim 15, wherein the applying energy comprises applying radio frequency energy to the shape memory material.

23. The method of claim 15, further comprising advancing a delivery catheter having distal and proximal ends and a lumen extending therethrough through said blood vessel proximal to said aneurysm, and wherein advancing said vascular stent further comprises advancing said vascular stent through said lumen of said delivery catheter.

24. The method of claim 23, wherein the advancing said vascular stent further comprises:
coupling the vascular stent to an elongate delivery member; and
advancing said delivery wire through said lumen of said delivery catheter.

25. The method of claim 23, wherein applying energy further comprises advancing a radio frequency probe through the delivery catheter and contacting the radio frequency probe to said vascular stent to deliver radio frequency energy to said shape memory material of at least one of said rings.

26. An adjustable shape-memory vascular stent, comprising:
a body having distal and proximal ends and comprising a shape memory material, said body having a first linear configuration and a second coiled configuration, said body being changeable from said first configuration to said second configuration in response to an application of an activation energy to said shape memory material.

27. The stent of claim 26, wherein said shape memory material has an austenite transition temperature of between about 38° C. and about 75° C., and wherein said application of said activation energy raises a temperature of said shape memory material, thereby resulting in said shape memory material changing from said first configuration to said second configuration.

28. The stent of claim 26, wherein said shape memory material has an austenite transition temperature of between about 39° C. and about 75° C., and wherein said application of energy that raises the temperature of said shape memory material to the austenite transition temperature results in said body changing from said first configuration to said second configuration.

29. The stent of claim 27, wherein said shape memory material has a starting austenite transition temperature of approximately 39° C. and a finish austenite transition temperature of approximately 75° C., and wherein application of energy that raises a temperature of said shape memory material above said starting austenite temperature results in said body changing from said first configuration to said second coiled configuration.

30. The stent of claim 29, wherein application of energy that raises the temperature of said shape memory material beyond said starting austenite temperature results in a cross-sectional diameter of second coiled configuration to enlarge.

31. The stent of claim 30, wherein the cross-sectional diameter of the coiled configuration may be selected by raising a temperature of the shape memory material a selected amount above the starting austenite transition temperature.

32. The stent of claim 26, wherein the cross-sectional diameter of said coiled configuration is greater than the cross-sectional diameter of said linear configuration.

33. The stent of claim 26, wherein the cross-sectional diameter of said coiled configuration is operably sized to engage the walls of a patient's blood vessel.

34. The stent of claim 26, wherein the cross-sectional diameter of said coiled configuration is between about 2 mm and about 4.5 mm.

35. The stent of claim 26, wherein the length of said coiled configuration is between about 10 mm and about 20 mm.

36. The stent of claim 26, wherein the second coiled configuration comprises a passageway extending therethrough.

37. The stent of claim 26, wherein application of a second activation energy to said shape memory material causes said body to change from said second configuration back substantially to said first configuration.

38. The stent of claim 37, wherein application of said second activation energy reduces said temperature of said shape memory temperature to a temperature below an austenite transition temperature for said shape memory material.

39. The stent of claim 26, further comprising a radio frequency electrode located at the proximal end of said body for receiving radio frequency energy.

40. The stent of claim 26, further comprising a release member located at the proximal end of said body, said release member being configured to releasably couple the body to a delivery device.

41. An adjustable shape memory vascular stent, comprising:
an elongate member with a plurality of rings spaced apart along the length of the elongate member, said rings comprising at least one shape memory material, said rings having a first compressed configuration and a second expanded configuration, said rings being changeable from said first configuration to said second configuration in response to an application of energy to said shape memory material.

42. The stent of claim 41, wherein said shape memory material has an austenite transition temperature of between about 38° C. to about 75° C. and wherein said application of energy raises the temperature of said shape memory material thereby causing said rings to change from said first configuration to said second configuration.

43. The stent of claim 41, wherein said shape memory material has an austenite transition temperature of between about 39° C. to about 75° C. and wherein said application of energy that raises a temperature of said shape memory material to the austenite transition temperature causes said rings to change from said first configuration to said second configuration.

44. The stent of claim 41, wherein the rings in said second configuration have a larger cross-sectional diameter than said rings in said first configuration.

45. The stent of claim 41, wherein each of said plurality of rings further comprises an radio frequency electrode for receiving energy.

46. The stent of claim 45, wherein said elongate member comprises an insulating material such that each of said rings is insulated from one another.

47. The stent of 45, wherein each of said rings comprises alternating segments of shape memory material and insulating material.

48. An adjustable shape-memory vascular stent, comprising:
means for stenting a blood vessel, said means comprising a shape memory material and having a first linear configuration and a second coiled configuration, said means for stenting being changeable from said first configuration to said second configuration in response to an application of an activation energy to said shape memory material.
